(19) United States
(12) Patent Application Publication
(10) Pub. No.: US 2024/0423586 A1
(43) Pub. Date: Dec. 26, 2024

(54) DISTAL END COMPONENT PORTION, ENDOSCOPE INSERTION PORTION, ENDOSCOPE AND ULTRASOUND ENDOSCOPE

(71) Applicant: OLYMPUS MEDICAL SYSTEMS CORP., Tokyo (JP)

(72) Inventor: Takahiro SUWA, Tokyo (JP)

(73) Assignee: OLYMPUS MEDICAL SYSTEMS CORP., Tokyo (JP)

(21) Appl. No.: 18/740,940

(22) Filed: Jun. 12, 2024

Related U.S. Application Data
(60) Provisional application No. 63/521,956, filed on Jun. 20, 2023.

(51) Int. Cl. A61B 8/12; A61B 8/00

(52) U.S. Cl. CPC A61B 8/12; A61B 8/4209; A61B 8/445

(57) ABSTRACT

A distal end component portion includes: a distal end portion configured to be arranged at a distal end of an insertion portion of the endoscope; and a channel communicating with a proximal end side of the distal end portion from an opening on an outer surface of the distal end portion, wherein the channel includes: a transition portion; a first portion extending distally from the transition portion along a first direction relative to the opening; and a second portion extending proximally from the transition portion along a second direction forming a first angle that is an obtuse angle to the first direction, and the first portion comprising a first recess having a first surface that recesses from the opening toward a first side opposite to a direction in which the first direction extends.

### CROSS-REFERENCE TO RELATED APPLICATION

This application is based on and claims priority under 35 U.S.C. § 119 to U.S. Provisional Application No. 63/521,956, filed Jun. 20, 2023, the entire contents of which are incorporated herein by reference.

### BACKGROUND

The present disclosure relates to a distal end component portion, an endoscope insertion portion, an endoscope, and an ultrasound endoscope.

A technique of injecting air or deaerated water into a balloon that is attached to a distal end of an insertion portion configured to be inserted into a subject and thus causing the balloon to adhere to an observed region, such as the digestive tract, has been known.

In Japanese Laid-open Patent Publication No. 2006-230950, a technique of injecting air into a balloon from a vent that is formed through a distal end rigid portion (distal end portion) to expand the balloon is described. It is also described that the inlet/outlet of the vent is oblique to the longitudinal direction of the insertion portion and therefore a brush for cleaning is easily inserted from the inlet/outlet of the vent.

The brush for cleaning is attached to the distal end of a long wire in order to clean through the duct communicating with the vent.

### SUMMARY

In some embodiments, a distal end component portion includes: a distal end portion configured to be arranged at a distal end of an insertion portion of the endoscope; and a channel communicating with a proximal end side of the distal end portion from an opening on an outer surface of the distal end portion, wherein the channel includes: a transition portion; a first portion extending distally from the transition portion along a first direction relative to the opening; and a second portion extending proximally from the transition portion along a second direction forming a first angle that is an obtuse angle to the first direction, and the first portion comprising a first recess having a first surface that recesses from the opening toward a first side opposite to a direction in which the first direction extends. In some embodiments, an endoscope insertion portion includes: the distal end component portion; and an insertion portion having a distal end at which the distal end component portion is arranged.

In some embodiments, an endoscope includes: the distal end component portion; and an insertion portion having a distal end at which the distal end component portion is arranged.

In some embodiments, an ultrasound endoscope includes: the distal end component portion; and an insertion portion having a distal end at which the distal end component portion is arranged. An ultrasound transducer is arranged at the distal end component portion.

The above and other features, advantages and technical and industrial significance of this disclosure will be better understood by reading the following detailed description of embodiments of the disclosure, when considered in connection with the accompanying drawings.

### BRIEF DESCRIPTION OF THE DRAWINGS

FIG. 1 is a diagram schematically illustrating an endoscopic system according to an embodiment;

FIG. 2 is an A-arrow view of FIG. 1;

FIG. 3 is a B-arrow view of FIG. 2;

FIG. 4 is a cross-sectional view of a distal end component portion orthogonal to its longitudinal direction;

FIG. 5 is a cross-sectional view of the distal end component portion along its longitudinal direction;

FIG. 6 is a diagram of a balloon liquid supply port viewed from a distal end side;

FIG. 7 is a schematic configuration diagram of a brush;

FIG. 8 is a cross-sectional view of a distal end component portion along its longitudinal direction according to Modification 1;

FIG. 9 is a diagram of a balloon water transfer port viewed from a distal-end side;

FIG. 10 is a cross-sectional view of a distal end component portion along its longitudinal direction according to Modification 2;

FIG. 11 is a cross-sectional view of a distal end component portion along its longitudinal direction according to Modification 3;

FIG. 12 is a partial enlarged view of an imaging unit;

FIG. 13 is a cross-sectional view of a distal end portion;

FIG. 14 is a partial enlarged view of a distal end portion of a treatment tool channel;

FIG. 15 is a cross-sectional view of a treatment tool channel block;

FIG. 16 is a perspective view of the treatment tool channel block; and

FIG. 17 is a cross-sectional view of the treatment tool channel block.

### DETAILED DESCRIPTION

Embodiments of a distal end component portion, an endoscope insertion portion, and an endoscope according to the disclosure will be described below with reference to the drawings. Note that the embodiments do not limit the disclosure. The disclosure is applicable to distal end component portions, endoscope insertion portions, and endoscopes generally.

In the illustration of the drawings, the same or corresponding components are denoted with the same reference numerals as appropriate. The drawings are schematic and it is necessary to note that the correlation among components in size and the ratio among the components may differ from actual ones. The drawings may contain portions whose correlation in size and whose ratio differ among the drawings.

### Embodiment

### Configuration of Endoscopic System

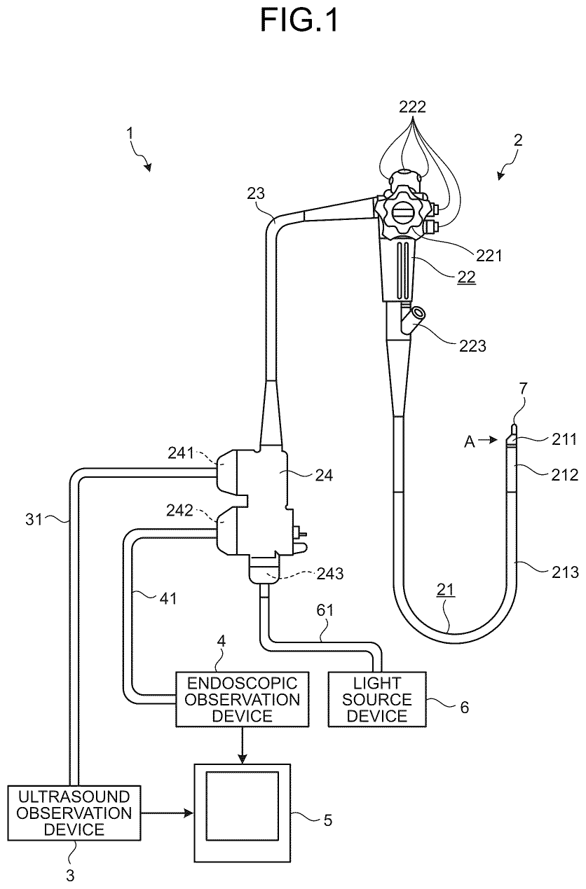

FIG. 1 is a diagram schematically illustrating an endoscopic system according to an embodiment. An endoscopic system 1 is a system that performs internal ultrasound diagnosis on a subject, such as a person, using an ultrasound endoscope. As illustrated in FIG. 1, the endoscopic system 1 includes an ultrasound endoscope 2, an ultrasound observation device 3, an endoscopic observation device 4, a display device 5, and a light source device 6.

Using an ultrasound transducer that is provided at a distal end portion of the ultrasound endoscope 2, the ultrasound endoscope 2 converts an electric pulse signal that is received from the ultrasound observation device 3 into ultrasound pulses and applies the ultrasound pulses to the subject and converts ultrasound echoes that are reflected by the subject into an electric echo signal expressing the ultrasound echoes by changes in voltage and outputs the echo signal.

The ultrasound endoscope 2 generally includes an imaging optical system and an imaging element and is inserted into a digestive tract (esophagus, stomach, duodenum or large intestine) or a respiratory organ (trachea or bronchi) of the subject and thus is capable of capturing images of the digestive tract or the respiratory organ. It is also possible to capture images of organs around the digestive tract or the respiratory organ (such as the pancreas, gallbladder, bile duct, biliary tract, lymph nodes, organ in the mediastinum, blood vessels) using ultrasound. The ultrasound endoscope 2 includes a light guide that guides illumination light that is applied to the subject when optical imaging is performed. While a distal end portion of the light guide reaches a distal end of the portion of the ultrasound endoscope 2 to be inserted into the subject, a proximal end portion of the light guide is connected to the light source device 6 that generates illumination light.

As illustrated in FIG. 1, the ultrasound endoscope 2 includes an insertion portion 21, an operation unit 22, a universal cord 23, and a connector 24.

The insertion portion 21 is a portion configured to be inserted into the subject. As illustrated in FIG. 1, the insertion portion 21 includes a distal end portion 211 that is rigid, that is provided on the side of a distal end of the ultrasound endoscope 2, and that holds an ultrasound transducer 7 that is of a convex type, a curving tube 212 that can curve in a curving direction orthogonal to a center axis, and an insertion tube 213 that is flexible and that has the center axis. The distal end portion 211 is provided on the side of a distal end of the curving tube 212, the curving tube 212 is provided on the side of a distal end of the insertion tube 213, and the insertion tube 213 is provided on the side of a distal end of the operation unit 22. A configuration of a distal end of the insertion portion 21 and a configuration of the curving tube 212 will be described below.

The operation unit 22 is a part that is joined to the side of a proximal end of the insertion portion 21 and that receives various types of operations from an operator, such as a doctor. As illustrated in FIG. 1, the operation unit 22 includes a curving knob 221 that receives a curving operation of curving the curving tube 212 and a plurality of operation portions 222 for performing various types of operations. In the operation unit 22, a treatment tool insertion port 223 that communicates with a treatment tool insertion path and that is for inserting the treatment tool into the treatment tool insertion path is formed.

The universal cord 23 is a cable that extends from the operation unit 22 and in which a plurality of signal cables that transmit various types of signals, an optical fiber that transmits illumination light that is supplied from the light source device 6, etc., are arranged.

The connector 24 is provided at a distal end of the universal cord 23. The connector 24 includes a first connector portion 241 to which an ultrasound cable 31 is connected, a second connector portion 242 to which a video cable 41 is connected, and a third connector portion 243 to which an optical fiber cable 61 is connected.

The ultrasound observation device 3 is electrically connected to the ultrasound endoscope 2 via the ultrasound cable 31 (refer to FIG. 1) and outputs the pulse signal to the ultrasound endoscope 2 via the ultrasound cable 31 and the echo signal is input to the ultrasound observation device 3 from the ultrasound endoscope 2. The ultrasound observation device 3 generates a ultrasound image by performing given processing on the echo signal.

The endoscopic observation device 4 is electrically connected to the ultrasound endoscope 2 via the video cable 41 (refer to FIG. 1) and an image signal from the ultrasound endoscope 2 is input to the endoscopic observation device 4 via the video cable 41. The endoscopic observation device 4 generates an endoscopic image by performing given processing on the image signal.

The display device 5 is configured using liquid crystals or EL (Electro Luminescence), a projector, a CRT (Cathode Ray Tube), or the like, and displays the ultrasound image that is generated by the ultrasound observation device 3 or the endoscopic image that is generated by the endoscopic observation device 4.

The light source device 6 is connected to the ultrasound endoscope 2 via the optical fiber cable 61 (refer to FIG. 1) and supplies the illumination light that illuminates the inside of the subject to the ultrasound endoscope 2 via the optical fiber cable 61.

### Configuration of Distal End Component Portion

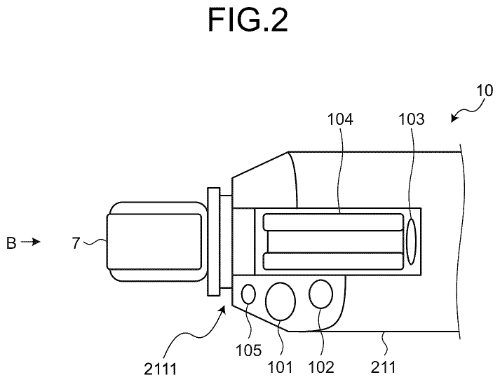

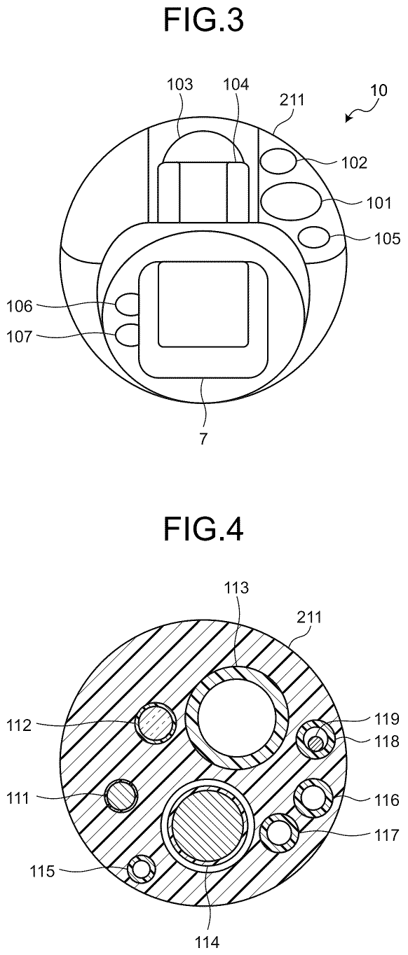

FIG. 2 is an A-arrow view of FIG. 1. FIG. 3 is a B-arrow view of FIG. 2. As illustrated in FIG. 2 and FIG. 3, at a distal end of the distal end portion 211 in a distal end component portion 10 that is arranged at a distal end of the insertion portion 21 of the ultrasound endoscope 2, an imaging lens 101 that focuses light from the subject, an illumination lens 102 that applies illumination light to the inside of the subject, a treatment tool protrusion port 103 that allows a treatment tool, such as forceps, to protrude, a treatment tool raising table 104 for raising the treatment tool upward in FIG. 3, a gas and liquid supply port 105 that is an opening that let a gas, such as the air, or a liquid, such as water, out, a balloon liquid supply port 106 that is an opening that supplies a liquid, such as water into the balloon, and a balloon suction port 107 that is an opening from which a liquid, such as water, is sucked from the inside of the balloon are arranged. The distal end portion 211 has a balloon groove 2111 on which a balloon band is caught and the balloon liquid supply port 106 is formed on the distal end side with respect to the balloon groove 2111.

### Internal Configuration of Distal End Component Portion

FIG. 4 is a cross-sectional view of the distal end component portion orthogonal to its longitudinal direction. As illustrated in FIG. 4, a signal cable 111 that transmits an image signal into which light that is received by the imaging lens 101 is converted, a light guide 112 that transmits illumination light to the illumination lens 102, a channel tube 113 into which the treatment tool is inserted, a transducer cable 114 that transmits and receives signals to and from the ultrasound transducer 7, a gas and liquid supply tube 115 that transmits a gas or a liquid to the gas and liquid supply port 105, a balloon liquid supply tube 116 that transmits a liquid to the balloon liquid supply port 106, a balloon suction tube 117 that transmits the liquid that is sucked from the balloon suction port 107, a treatment tool raising tube 118 into which a treatment tool raising wire 119 is inserted, and the treatment tool raising wire 119 that causes the treatment tool raising table to be raised according to an operation on the operation unit 22 are inserted into the distal end portion 211.

### Configuration of Balloon Liquid Supply Channel

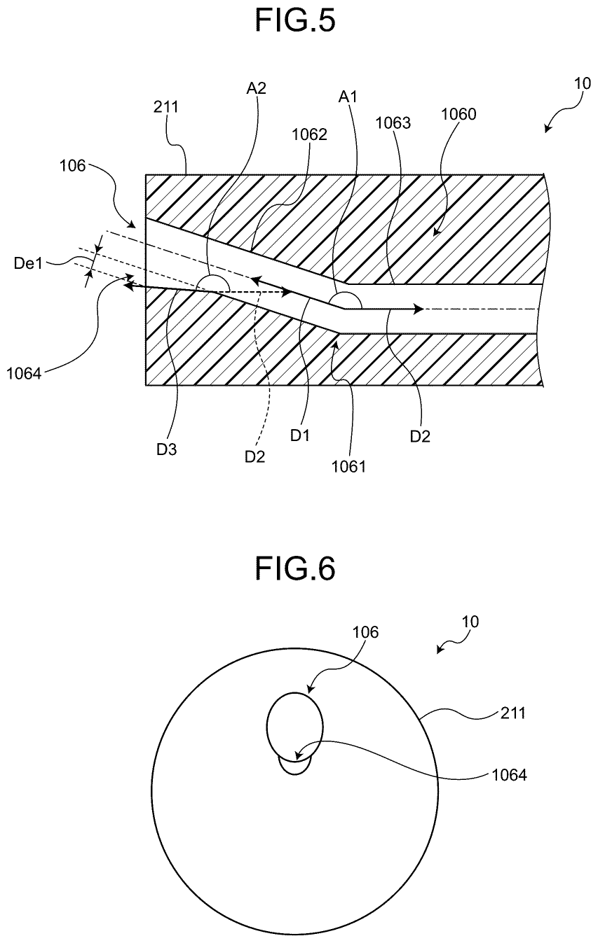

FIG. 5 is a cross-sectional view of the distal end component portion along its longitudinal direction. As illustrated in FIG. 5, the distal end component portion 10 includes the distal end portion 211 that is arranged at the distal end of the insertion portion 21 of the ultrasound endoscope 2 and a balloon liquid supply channel (channel) 1060 that is a fluid channel that communicates with a proximal end side of the distal end portion 211 from the balloon liquid supply port 106 formed on an outer surface of the distal end portion 211 and that includes a curve portion (transition portion) 1061 that is provided at a predetermined position from the balloon liquid supply port 106 toward the proximal end side and to which the balloon liquid supply tube 116 is connected on the proximal end side.

The balloon liquid supply channel 1060 includes a first portion 1062 that extends from the curve portion 1061 toward the distal end side to the balloon liquid supply port 106 along a first direction D1 and a second portion 1063 that extends from the curve portion 1061 toward the proximal end side along a second direction D2 forming a first angle A1 that is an obtuse angle with the first direction D1. The second direction D2 is a direction along a longitudinal direction of the insertion portion 21; however, the second direction D2 may be a direction intersecting the longitudinal direction of the insertion portion 21.

The distal end portion 211 has a distal end surface intersecting the longitudinal direction of the insertion portion 21 and a side surface along the longitudinal direction and the balloon liquid supply port 106 is formed in the distal end surface. In the distal end portion 211, a first recess 1064 that recesses from the balloon liquid supply port 106 toward a side opposite to a direction (upper side in FIG. 5) in which the first direction D1 curves with respect to the second direction D2 is formed. The first recess 1064 has a first surface. The first surface has a slope extending toward the distal end side in a third direction D3 forming a second angle A2 larger than the first angle A1 with the second direction D2. A depth Del by which the first recess 1064 recesses is, for example, 0.3 mm or larger. Note that the first recess 1064 may be a diameter-increased portion that increases a diameter of a circular shape.

FIG. 6 is a diagram of the balloon liquid supply port viewed from the distal end side. As illustrated in FIG. 6, the balloon liquid supply port 106 has a circular shape. The first recess 1064 has a semicircular shape whose diameter is smaller than that of the circular shape of the balloon liquid supply port 106.

### Configuration of Brush

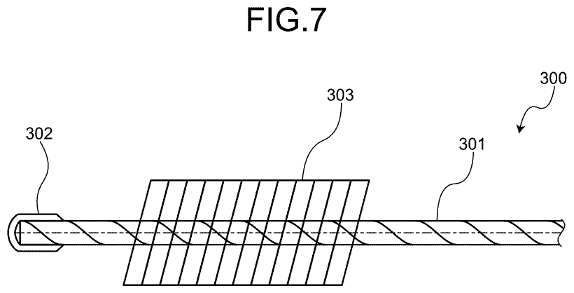

FIG. 7 is a schematic configuration diagram of a brush. It is possible to clean the entire balloon liquid supply channel 1060 by inserting a brush 300 illustrated in FIG. 7 from the side of the operation unit 22 and causing the brush 300 to protrude from the balloon liquid supply port 106. The brush 300 includes a wire 301, a distal end chip 302, and a brush part 303. The distal end chip 302 is formed by swaging a metal part to a distal end of the wire 301. As a result, the distal end chip 302 forms a step in a direction orthogonal to a longitudinal direction of the wire 301. The height of the step in a direction orthogonal to the longitudinal direction of the wire 301 is smaller than 0.3 mm.

According to the distal end component portion 10 described above, because the first recess 1064 is formed in the distal end portion 211, a surface of the distal end chip 302 of the brush 300 caused to protrude from the balloon liquid supply port 106 on the proximal end side is prevented from getting caught on the edge of the balloon liquid supply port 106 and damaging the distal end portion 211.

Furthermore, because the depth Del of the first recess 1064 is set at 0.3 mm or larger and thus the depth Del of the first recess 1064 is larger than the height of the step formed by the distal end chip 302, it is possible to assuredly prevent the surface of the distal end chip 302 of the brush 300 that is caused to protrude from the balloon liquid supply port 106 on the proximal end side from getting caught on the edge of the balloon liquid supply port 106.

In addition to the balloon liquid supply port 106, the same configuration as that of the embodiment may be employed for the gas and liquid supply port 105 or the balloon suction port 107. Also in this case, it is possible to prevent the distal end portion from being damaged by the wire of the brush for cleaning.

The distal end portion 211 may have a distal end surface intersecting with the longitudinal direction of the insertion portion 21 and a side surface along the longitudinal direction and the balloon liquid supply port 106 may be formed on a side surface. The distal end portion 211 may have a first balloon groove on which a balloon band on the proximal end side is caught and a second balloon groove that is positioned on the distal end side with respect to the first balloon groove and on which a balloon band on the distal end side is caught and the balloon liquid supply port 106 may be formed between the first balloon groove and the second balloon groove.

### Modification 1

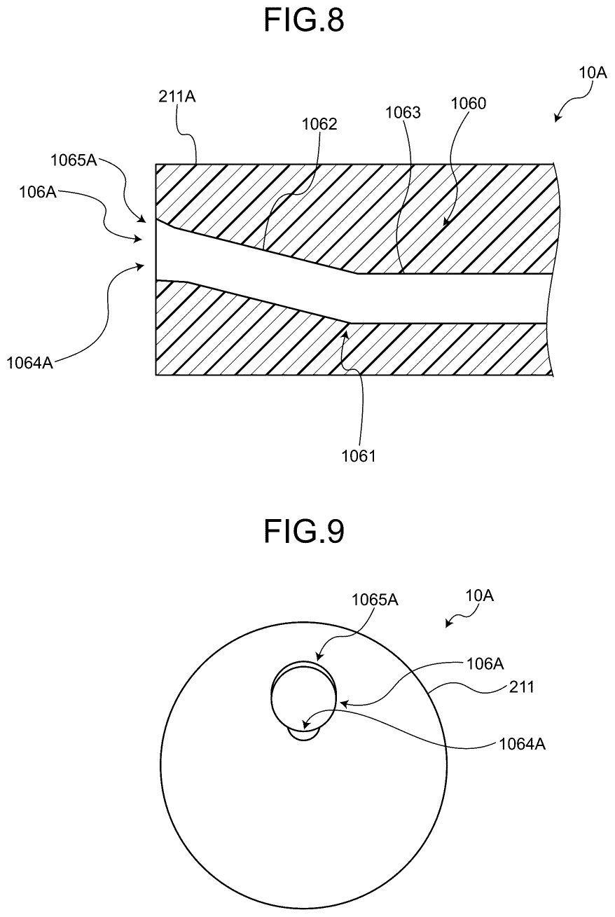

FIG. 8 is a cross-sectional view of a distal end component portion along its longitudinal direction according to Modification 1. FIG. 9 is a diagram of a balloon liquid supply port viewed from the distal end side. As illustrated in FIG. 8 and FIG. 9, in a distal end portion 211A of a distal end component portion 10A, a first recess 1064A that recesses from the balloon liquid supply port 106A to a side opposite to a direction (upper side in FIG. 8) in which a first direction curves with respect to a second direction is formed. Furthermore, in the distal end portion 211A, a second recess 1065A having a second surface that recesses from the balloon liquid supply port 106A in the direction in which the first direction curves with respect to the second direction is formed.

According to the distal end component portion 10A described above, because the first recess 1064A is formed in the distal end portion 211A, the surface of the distal end chip 302 of the brush 300 caused to protrude from the balloon liquid supply port 106A on the proximal end side is prevented from getting caught on the lower edge of the balloon liquid supply port 106A and damaging the distal end portion 211A.

Furthermore, according to the distal end component portion 10A, because the second recess 1065A is formed in the distal end portion 211A, the surface of the distal end chip 302 of the brush 300 caused to protrude from the balloon liquid supply port 106A on the proximal end side is prevented from getting caught on the upper edge of the balloon liquid supply port 106A and damaging the distal end portion 211A.

### Modification 2

FIG. 10 is a cross-sectional view of a distal end component portion along its longitudinal direction according to Modification 2. As illustrated in FIG. 10, in a distal end portion 211B of a distal end component portion 10B, a first recess 1064B that recesses from a balloon liquid supply port 106B to a side opposite to a direction (upper side in FIG. 10) in which a first direction curves with respect to a second direction is formed. The first recess 1064B has a smooth curve surface forming a round shape toward a distal end side. Furthermore, in the distal end portion 211B, a second recess 1065B that recesses from the balloon liquid supply port 106B in a direction in which the first direction curves with respect to the second direction is formed.

According to the distal end component portion 10B described above, because the first recess 1064B is formed in the distal end portion 211B, the surface of the distal end chip 302 of the brush 300 caused to protrude from the balloon liquid supply port 106B on the proximal end side is prevented from getting caught on the lower edge of the balloon liquid supply port 106B and damaging the distal end portion 211B.

Furthermore, according to the distal end component portion 10B, because the second recess 1065B is formed in the distal end portion 211B, the surface of the distal end chip 302 of the brush 300 caused to protrude from the balloon liquid supply port 106B on the proximal end side is prevented from getting caught on the upper edge of the balloon liquid supply port 106B and damaging the distal end portion 211B.

### Modification 3

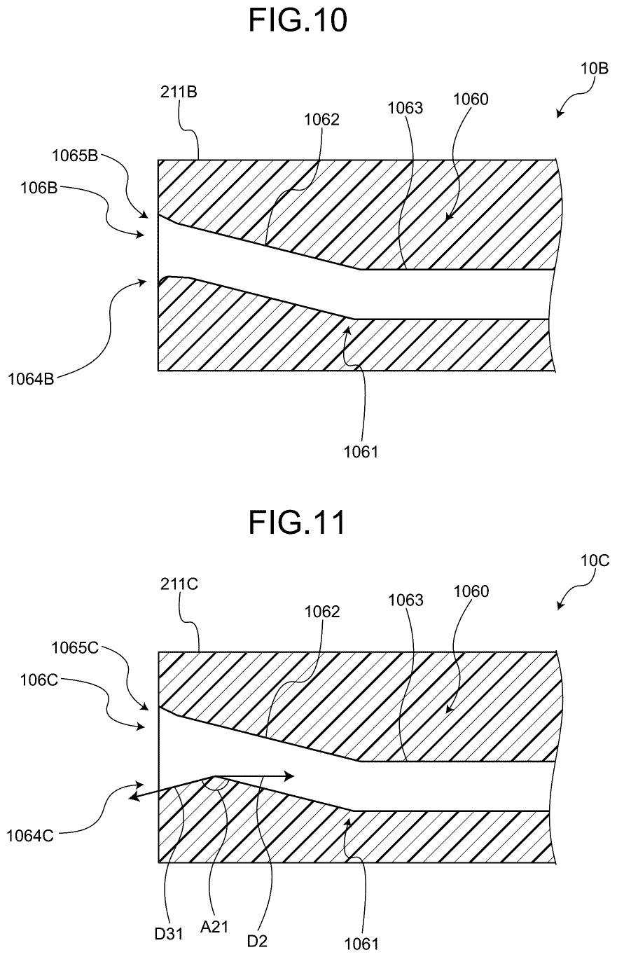

FIG. 11 is a cross-sectional view of a distal end component portion along its longitudinal direction according to Modification 3. As illustrated in FIG. 11, in a distal end portion 211C of a distal end component portion 10C, a first recess 1064C that recesses from a balloon liquid supply port 106C to a side opposite to a direction (upper side in FIG. 11) in which a first direction curves with respect to a second direction is formed. The first recess 1064C has a slope extending in a third direction D31 forming a second angle A21 larger than the first angle A1 with the second direction D2 toward the distal end side. Note that the second angle A21 is a smaller angle among a counterclockwise angle from the second direction D2 toward a third direction D31 and a clockwise angle from the second direction D2 to the third direction D31. When the second angle A21 is a clockwise angle from the second direction D2 to the third direction D31 as in Modification 3, the angle can be an angle satisfying 90°<second angle A21≤180°. Furthermore, in the distal end portion 211C, a second recess 1065C that recesses from the balloon liquid supply port 106C in a direction in which the first direction curves with respect to the second direction is formed.

According to the distal end component portion 10C described above, because the first recess 1064C is formed in the distal end portion 211C, the surface of the distal end chip 302 of the brush 300 caused to protrude from the balloon liquid supply port 106C on the proximal end side is prevented from getting caught on the lower edge of the balloon liquid supply port 106C and damaging the distal end portion 211C.

Furthermore, according to the distal end component portion 10C, because the second recess 1065C is formed in the distal end portion 211C, the surface of the distal end chip 302 of the brush 300 caused to protrude from the balloon liquid supply port 106C on the proximal end side is prevented from getting caught on the upper edge of the balloon liquid supply port 106C and damaging the distal end portion 211C.

### Configuration of Imaging Unit

FIG. 12 is a partial enlarged view of an imaging unit. As illustrated in FIG. 12, the imaging unit includes the imaging lens 101 that is positioned at a distal end, an imaging unit 121 including an imaging element that converts light that is focused by the imaging lens 101 into an image signal, and a signal cable 111 that transmits the image signal that is generated by the imaging element to the endoscopic observation device 4.

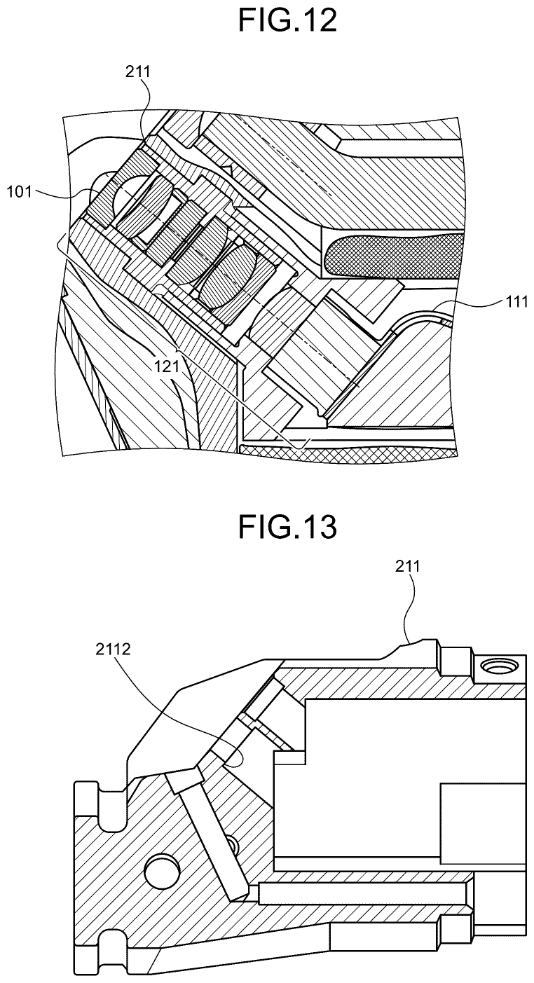

FIG. 13 is a cross-sectional view of the distal end portion. As illustrated in FIG. 13, an abutting portion 2112 is formed in the distal end portion 211. The abutting portion 2112 is formed by grinding a step from the distal end side by boring. The imaging unit 121 is made abut against the abutting portion 2112 and is fixed and the imaging lens 101 is attached, so that the imaging unit is formed.

On the other hand, when boring is performed from the proximal end side, a cover structure may be employed to cover the processing hole because a processing hole is made on a side surface. The cover structure is not preferable from a standpoint of reprocessing and costs and therefore boring from the distal end side makes it possible to avoid the cover structure.

### Configuration of Treatment Tool Channel

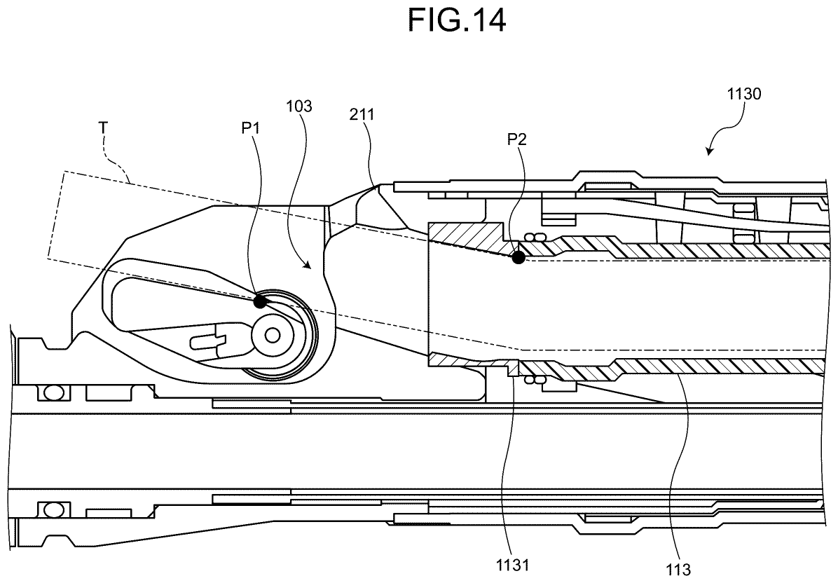

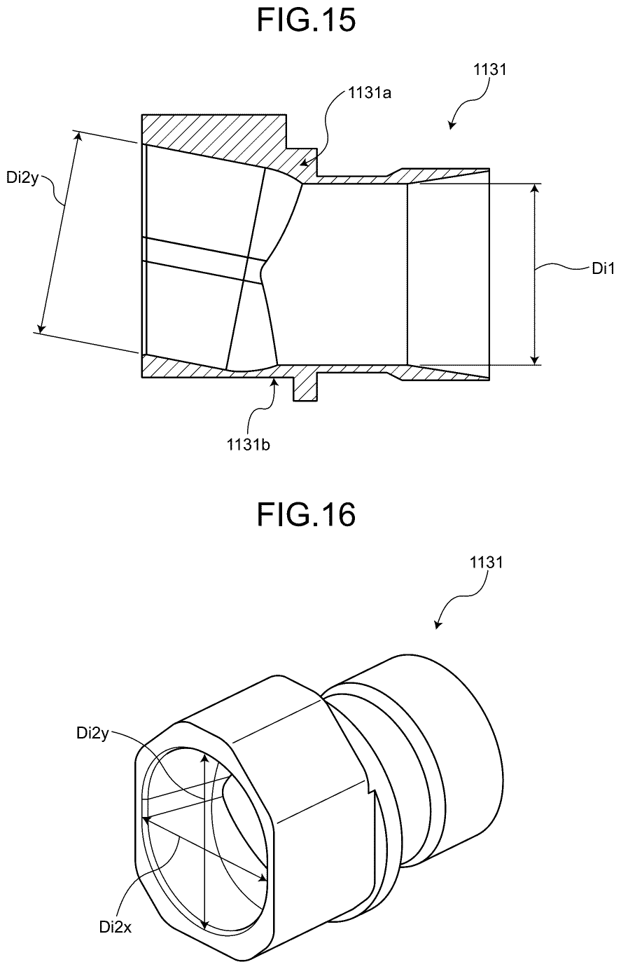

FIG. 14 is a partial enlarged view of a distal end portion of a treatment tool channel. As illustrated in FIG. 14, a treatment tool channel 1130 includes a treatment tool channel block 1131 that is arranged on the proximal end side of the treatment tool protrusion port 103 and a channel tube 113 that is connected to the proximal end side of the treatment tool channel block 1131.

FIG. 15 is a cross-sectional view of the treatment tool channel block. As illustrated in FIG. 15, the treatment tool channel block 1131 is provided with diameter-increased portions 1131a and 1131b whose inner diameters are increased more than that of the proximal end side of a slope that slopes toward the treatment tool protrusion port 103. As a result, an inner diameter Di2y on the distal end side of the slope is larger than an inner diameter Di1 of the proximal end side of the slope.

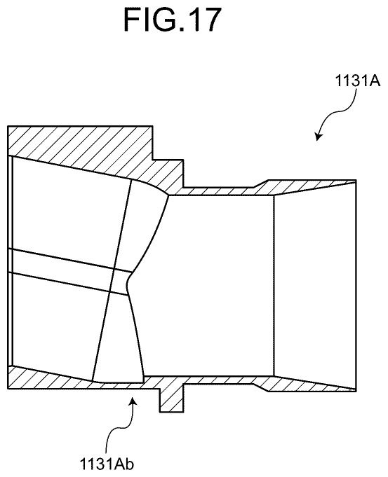

FIG. 16 is a perspective view of the treatment tool channel block. As illustrated in FIG. 16, the diameter-increased portions 1131a and 1131b are provided in a vertical direction (y-direction) and therefore an inner diameter Di2y in the vertical direction is larger than an inner diameter Di2x in the horizontal direction (x-direction). As a result, the distal end side of the treatment tool channel block 1131 has an oval opening.

Back to FIG. 14, when a thick treatment tool T is inserted into the treatment tool channel 1130 and is raised, a point P1 servers as a point of effort and a point P2 serves as a fulcrum. The position of the fulcrum P2 shifts to the distal end side because of the diameter-increased portion 1131a. As a result, the point P1 serving as the point of effort and the point P2 serving as the fulcrum get close in distance to each other and a reaction force decreases and therefore a force required to raise the treatment tool T is reduced. Furthermore, because the positon of the fulcrum P2 shifts to the distal end side, it is possible to increase an angle at which the treatment tool T is raised.

Furthermore, because the diameter increased portion 1131b is formed, when the treatment tool T is inserted into the treatment tool channel block 1131, the diameter increased portion 1131b is connected to the proximal end side of the slope at an angle larger than 180°, which prevents the treatment tool T from getting caught on the slope.

FIG. 17 is a cross-sectional view of the treatment tool channel block. As illustrated in FIG. 17, a treatment tool channel block 1131A may have a step 1131Ab whose inner diameter is increased more than that on the distal end side of the slope sloping toward the treatment tool protrusion port 103. The step 1131Ab is formed and therefore, when the treatment tool T is inserted into the treatment tool channel block 1131, the treatment tool T is prevented from getting caught on the slope.

According to the disclosure, it is possible to realize a distal end component portion, an endoscope insertion portion, and an endoscope in which a distal end portion is prevented from being damaged by a wire of the brush for cleaning.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A distal end component portion for use with an endoscope, the distal end component portion comprising:
a distal end portion configured to be arranged at a distal end of an insertion portion of the endoscope; and
a channel communicating with a proximal end side of the distal end portion from an opening on an outer surface of the distal end portion,
wherein the channel includes:
a transition portion;
a first portion extending distally from the transition portion along a first direction relative to the opening; and
a second portion extending proximally from the transition portion along a second direction forming a first angle that is an obtuse angle to the first direction, and
the first portion comprising a first recess having a first surface that recesses from the opening toward a first side opposite to a direction in which the first direction extends.

2. The distal end component portion according to claim 1, wherein the first surface has a slope that extends toward a distal end side in a third direction forming a second angle larger than the first angle with the second direction.

3. The distal end component portion according to claim 1, wherein the second direction is a direction along a longitudinal direction of the insertion portion.

4. The distal end component portion according to claim 1, wherein the opening has a circular shape, and
the first surface increases a diameter of the circular shape.

5. The distal end component portion according to claim 1, wherein the opening has a circular shape, and
the first surface has a semicircular shape whose diameter is smaller than that of the circular shape.

6. The distal end component portion according to claim 1, wherein a depth in which the first surface recesses from the opening is 0.3 mm or larger.

7. The distal end component portion according to claim 1, wherein the first portion further comprising a second recess having a second surface that recesses from the opening towards a second side in the direction in which the first direction extends.

8. The distal end component portion according to claim 1, wherein the distal end portion has a distal end surface intersecting a longitudinal direction of the insertion portion and a side surface along the longitudinal direction, and
the opening is in the distal end surface.

9. The distal end component portion according to claim 8, wherein the distal end portion includes a balloon groove for holding a balloon band, and
the opening is located distally relative to the balloon groove.

10. The distal end component portion according to claim 1, wherein the distal end portion has a distal end surface intersecting a longitudinal direction of the insertion portion and a side surface along the longitudinal direction, and
the opening is on the side surface.

11. The distal end component portion according to claim 10, wherein the distal end portion has a first balloon groove for holding a first balloon band on the proximal end side, and
the opening is located proximally relative to the first balloon groove.

12. An endoscope insertion portion comprising:
the distal end component portion according to claim 1; and
the insertion portion having the distal end at which the distal end component portion is arranged.

13. An endoscope comprising:
the distal end component portion according to claim 1; and
the insertion portion having the distal end at which the distal end component portion is arranged.

14. An ultrasound endoscope comprising:
the distal end component portion according to claim 1; and
the insertion portion having the distal end at which the distal end component portion is arranged,
wherein the distal end component further comprises an ultrasound transducer.

15. The distal end component portion according to claim 1,
wherein the channel has a first dimension at the opening along a direction orthogonal to a longitudinal direction of the insertion portion,
wherein the channel has a second dimension at a position located proximally relative to the opening along the direction, and
wherein the first dimension is larger than the second dimension.

16. The distal end component portion according to claim 1,
wherein the channel has a first dimension at the opening along a direction orthogonal to a longitudinal direction of the insertion portion,
wherein the channel has a second dimension at the transition portion along the direction, and
wherein the first dimension is larger than the second dimension.

17. The distal end component portion according to claim 1,
wherein the channel has a dimension along a direction orthogonal to a longitudinal direction of the insertion portion, the dimension decreasing toward the proximal end side.
